(19) United States
(12) Patent Application Publication
(10) Pub. No.: US 2024/0245585 A1
(43) Pub. Date: Jul. 25, 2024

(54) Dental Cement Composition, Kit of Parts and Use Thereof

(71) Applicant: 3M INNOVATIVE PROPERTIES COMPANY, St. Paul, MN (US)

(72) Inventors: Bernhard C. Hofmann, Peißenberg (DE); Reinhold Hecht, Schondorf am Ammersee (DE); Wolf Steiger, Weil-Petzenhausen (DE); Peter Bissinger, Diessen (DE)

(21) Appl. No.: 18/574,390

(22) PCT Filed: Jun. 1, 2022
(86) PCT No.: PCT/IB2022/055132

§ 371 Date: Dec. 27, 2023

(30) Foreign Application Priority Data
Jun. 28, 2021 (EP) 21181924.8

(51) Int. Cl. A61K 6/887; A61K 6/62; A61K 6/71; A61K 6/76; A61K 6/77; A61K 6/79; A61K 6/893; C04B 26/06; C04B 111/00

(52) U.S. Cl. CPC A61K 6/887; A61K 6/62; A61K 6/71; A61K 6/76; A61K 6/77; A61K 6/79; A61K 6/893; C04B 26/06; C04B 2111/00836

(57) ABSTRACT

The invention relates to a dental cement composition comprising a resin matrix comprising a (meth)acrylate component comprising at least two (meth)acrylate moieties, a polyol spacer group between the at least two (meth)acrylate moieties, the polyols spacer group having a molecular weight Mw of at least 3,000 g/mol, an acidic polymerizable component, preferably an acidic (meth)acrylate component, a non-acidic polymerizable component having a molecular weight Mw below 1,000 g/mol, preferably a non-acidic (meth)acrylate component, filler, an initiator system, optionally additives. The invention also relates to a kit of parts comprising the dental cement composition, a dental crown or a curable dental composition for producing a temporary dental restoration being different from the dental cement composition. The dental cement composition is intended for use in a process of temporarily restoring a dental situation in a mouth of a patient.

### FIELD OF THE INVENTION

The invention relates to dental cement composition, in particular a dental cement composition for temporary use. The dental cement composition comprises a (meth)acrylate component having a high molecular weight.

The invention relates also to a kit of parts comprising such a dental cement composition and the dental cement composition for use in a process of temporarily fixing a dental restoration to a dental situation in the mouth of a patient.

### BACKGROUND

Temporary dental cements are used for the fixing of a temporary dental restoration to a prepared tooth surface in the mouth of a patient for an interim period, i.e. for a time period until the permanent dental restoration is available.

To fulfil this function, the adhesion of the temporary dental cement to the prepared tooth surface may not be too high. Otherwise it may be difficult for the dental practitioner to remove the temporary dental restoration.

Meanwhile a variety of temporary dental cements are commercially available. Some of them are based on zinc oxide and contain eugenol or are eugenol free.

Some patients report that eugenol based temporary cements have an unpleasant smell. Further, residues of the eugenol based temporary cement on the prepared tooth surface may inhibit the curing reaction of dental resin cements which are used later for fixing a permanent restoration.

Known are also temporary dental cements which are based on (meth)acrylate chemistry.

However, upon removal eugenol free and (meth)acrylate-based dental cements often show a non-defined fracture pattern on the tooth surface, meaning that cement residues during the removal of the restoration remain both on the tooth surface and within the restoration. This results in a time-consuming cleaning procedure of the tooth surface to enable a successful permanent cementation later. In addition, also the sealing properties of (meth)acrylate based temporary cements are sometimes considered to be poor allowing contaminates to infiltrate the interface between the prepared tooth surface and the dental cement during the wearing time of the temporary restoration.

US2014/0213686A1 (Falsafi et al.) describes a dental cement composition for forming a temporary bond, comprising: a first part, the first part including water, an initiator, a reducing agent and a first filler; a second part, the second part including a monomer having at least one ethylenically unsaturated group per monomer molecule, an oxidizing agent and a second filler; and an adhesion reducing component of a polyhydric alcohol present in one of the first part or the second part of the dental cement composition for forming the temporary bond.

US2017/0014312A1 (Suzuki) describes a dental composition comprising (a) a (meth)acrylic acid ester polymer comprising a (meth)acrylic acid ester homopolymer having a glass transition temperature of 25° C. to 50° C., a random copolymer of a (meth)acrylic acid ester whose homopolymer has a glass transition temperature higher than 37° C., or both, and a (meth)acrylic acid ester whose homopolymer has a glass transition temperature lower than 37° C., the (meth)acrylic acid ester polymer (a) having a glass transition temperature of 25° C. to 50° C., having tan δ at 37° C. of 0.10 or more as determined by dynamic mechanical analysis, and having no melting point; (b) a polymerizable monomer comprising a (meth)acrylic acid ester, a (meth)acrylamide, or both; and (c) a polymerization initiator.

EP0988851A2 (Kerr) describes a transparent, elastomeric temporary dental cement composition comprising: (a) at least one binder selected from the group consisting of a multifunctional oligomer and a prepolymer; (b) at least one finely divided filler; and (c) at least one initiation system for polymerization. US2012/213832A1 (Ori et al.) describes a dental curable composition comprising (A) a polymerizable monomer and (B) an organic amine-based polymerization initiator wherein (A′) a long-chained polymerizable monomer having a chain length of 17 or more atoms is contained in the component (A) and/or (C) a soft resin material are/is contained in the composition. The curable composition is said to be easily removable.

### SUMMARY OF INVENTION

There is still a need for an alternative or improved temporary dental cement composition.

The dental cement composition should have sufficient adhesion to a prepared tooth surface.

The dental cement composition should also have sufficient adhesion to the temporary dental restoration.

Ideally, the dental cement composition should be easy to remove from the prepared tooth surface and stay in the temporary restoration, if desired.

If possible, the dental cement composition should have good flowing properties.

Further, if possible, the dental cement composition should be essentially free of softeners.

In one embodiment the present invention features a dental cement composition comprising
a resin matrix comprising
   a (meth)acrylate component comprising
      at least two (meth)acrylate moieties,
      a polyol spacer group between the at least two (meth)acrylate moieties, the polyols spacer group having a molecular weight Mw of at least 2,500 g/mol, preferably of at least 3,000 g/mol,
   an acidic polymerizable component, preferably an acidic (meth)acrylate component,
   a non-acidic polymerizable component having a molecular weight Mw below 1,000 g/mol, preferably a non-acidic (meth)acrylate component, filler,
an initiator system,
optionally additives.

In another embodiment, the invention relates to a kit of parts comprising the dental cement composition as described in the present text, and a dental crown or a curable dental composition for producing a temporary dental restoration being different from the dental cement composition.

A further embodiment of the invention is directed to a dental cement composition as described in the present text for use in a process of restoring a dental situation in the mouth of a patient.

Unless defined differently, for this description the following terms shall have the given meaning:

“One component composition” means that all of the components mentioned are present in the composition during storage and use. That is, the composition to be applied or used is not prepared by mixing different parts of the composition before use. In contrast to one-component compositions, those compositions are often referred to as two-component compositions (e.g. being formulated as powder/liquid, liquid/liquid or paste/paste compositions).

“Two component composition” means that the components are provided as a kit of parts or system in parts separated from each other before use. For use, the respective components or parts need to be mixed.

A “dental composition” or a “composition for dental use” or a “composition to be used in the dental field” is any composition which can be used in the dental field. In this respect the composition should be not detrimental to the patients' health and thus be free of hazardous and toxic components being able to migrate out of the composition. Examples of dental compositions include permanent and temporary crown and bridge materials, artificial crowns, anterior or posterior filling materials, adhesives, mill blanks, lab materials, luting agents and orthodontic devices. Dental compositions are typically hardenable compositions, which can be hardened at ambient conditions, including a temperature range from 15 to 50° C. or from 20 to 40° C. within a time frame of 30 min or 20 min or 10 min. Higher temperatures are not recommended as they might cause pain to the patient and may be detrimental to the patient's health. Dental compositions are typically provided to the practitioner in comparable small volumes, that is volumes in the range from 0.1 to 100 ml or from 0.5 to 50 ml or from 1 to 30 ml. Thus, the storage volume of useful packaging devices is within these ranges.

The term “temporary cement” refers to a cement which can hold a dental material, such as a dental prosthetic device, for example, a crown, in place on a dental structure, such as a prepared tooth or implant, under normal oral conditions of use for the service life of the device, and further, which facilitates easy removal of the device when needed without damage to the tooth or implant. Such temporary cements, therefore, allow easy removal. Temporary cements are typically used for a period of 1 day to 6 months.

The term “compound” or “component” is a chemical substance which has a particular molecular identity or is made of a mixture of such substances, e.g., polymeric substances.

A “monomer” is any chemical substance which can be characterized by a chemical formula, bearing polymerizable groups (including (meth)acrylate groups) which can be polymerized to oligomers or polymers or crosslinked networks thereby increasing the molecular weight. The molecular weight of monomers can usually simply be calculated based on the chemical formula given.

As used herein, “(meth)acryl” is a shorthand term referring to “acryl” and/or “methacryl”. For example, a “(meth) acryloxy” group is a shorthand term referring to either an acryloxy group (i.e., CH₂═CH—C(O)—O—) and/or a methacryloxy group (i.e., CH₂═C(CH₃)—C(O)—O—). Similarly, (meth)acrylate is a shorthand term referring to “acrylate” and/or “methacrylate.”

A “hardenable component or material” or “polymerizable component” is any component which can be cured or solidified e.g. by heating to cause polymerization, chemical crosslinking, radiation-induced polymerization or crosslinking by using a redox initiator. A hardenable component may contain only one, two, three or more polymerizable groups. Typical examples of polymerizable groups include unsaturated carbon groups, such as a vinyl group being present i.a. in a (meth)acrylate group.

An “acidic polymerizable component” or an “ethylenically unsaturated acidic component” is meant to include monomers, oligomers, and polymers having ethylenic unsaturation and acid and/or acid-precursor functionality. Acidic precursor functionalities include, e.g. anhydrides, acid halides and pyrophosphates. The acidic group preferably comprises one or more carboxylic acid residues, such as —COOH or —CO—O—CO—, phosphoric acid residues, such as —O—P(O)(OH)OH, phosphonic acid residues such as C—P(O)(OH)OH, sulfonic acid residues, such as —SO₃H or sulfinic acid residues such as —SO₂H.

A “filler” contains all fillers being present in the hardenable composition. Only one type of filler or a mixture of different fillers can be used.

A “solvent” means a liquid which is able to at least partially disperse or dissolve a component at ambient conditions (e.g. 23° C.). A solvent typically has a viscosity below about 5 or below about 1 or below about 0.1 Pa*s.

By “paste” is meant a soft, viscous mass of solids (i.e. particles) dispersed in a liquid.

A “particle” means a substance being a solid having a shape which can be geometrically determined. The shape can be regular or irregular. Particles can typically be analysed with respect to e.g. grain size and grain size distribution.

An “adhesive” or “dental adhesive” refers to a composition used as a pre-treatment on a dental structure (e.g., a tooth) to adhere a “dental material” (e.g., “restorative” an orthodontic appliance (e.g., bracket), or an “orthodontic adhesive”) to a dental surface. An “orthodontic adhesive” refers to a composition used to adhere an orthodontic appliance to a dental (e.g., tooth) surface. Generally, the dental surface is pre-treated, e.g., by etching, priming, and/or applying an adhesive to enhance the adhesion of the “orthodontic adhesive” to the dental surface.

A “dental surface” or “tooth surface” refers to the surface of tooth structures (e.g., enamel, dentin, and cementum) and bone.

A “self-adhesive” composition refers to a composition that is capable of bonding to a dental surface without pre-treating the dental surface with a primer or bonding agent. Preferably, a self-adhesive composition is also a self-etching composition wherein no separate etchant is used.

A “self-curing composition” means a composition which cures by a redox-reaction without application of radiation.

“Radiation curable” shall mean that the component (or composition, as the case may be) can be cured by applying radiation, preferably electromagnetic radiation with a wavelength in the visible light spectrum under ambient conditions and within a reasonable time frame (e.g. within about 60, 30 or 10 seconds).

“Ambient conditions” mean the conditions which the composition described in the present text is usually subjected to during storage and handling. Ambient conditions may, for example, be a pressure of 900 to 1,100 mbar, a temperature of 10 to 40° C. and a relative humidity of 10 to 100%. In the laboratory ambient conditions are typically adjusted to 20 to 25° C. and 1,000 to 1,025 mbar with respect to maritime level.

A composition is “essentially or substantially free of” a certain component, if the composition does not contain said component as an essential feature. Thus, said component is not willfully added to the composition either as such or in combination with other components or ingredient of other components. A composition being essentially free of a certain component usually does not contain that component at all. However, sometimes the presence of a small amount of the said component is not avoidable e.g. due to impurities contained in the raw materials used.

As used herein, “a”, “an”, “the”, “at least one” and “one or more” are used interchangeably. Also herein, the recitations of numerical ranges by endpoints include all numbers subsumed within that range (e.g., 1 to 5 includes 1, 1.5, 2, 2.75, 3, 3.80, 4, 5, etc.).

Adding an “(s)” to a term means that the term should include the singular and plural form. E.g. the term “additive(s)” means one additive and more additives (e.g. 2, 3, 4, etc.).

Unless otherwise indicated, all numbers expressing quantities of ingredients, measurement of physical properties such as described below and so forth used in the specification and claims are to be understood as being modified in all instances by the term “about”.

The terms “comprise” or “contain” and variations thereof do not have a limiting meaning where these terms appear in the description and claims. “Consisting essentially of” means that specific further components can be present, namely those which do not materially affect the essential characteristic of the article or composition. “Consisting of” means that no further components should be present. The term “comprise” shall include also the terms “consist essentially of” and “consists of”.

### DESCRIPTION OF FIGURES

FIG. 1 shows the inner surface of a composite crown after a pull-off test where the dental cement composition described in the present text was used.

### DETAILED DESCRIPTION

The dental cement composition described in the present text has a couple of advantageous properties.

The dental cement composition shows adequate adhesion properties to dentin and to a temporary dental restoration, wherein the adhesion to the temporary dental restoration is higher than to dentin. This allows an easier removal of a temporary dental restoration from the prepared tooth surface.

The dental cement composition also has an adequate tensile strength which reduces the risk of a fracturing of the hardened composition during the removal process.

Thus, after removal of the temporary restoration the dental cement composition mainly remains in the temporary restoration and such simplifies the clean-up procedure of the tooth surface prior to the permanent cementation of the final restoration.

If desired, this can be evaluated by conducting a so-called crown pull-off test.

The dental cement composition also shows an adequate flexibility which can be determined by the elongation at break. Due to the presence of a high molecular weight (meth)acrylate component, the dental cement composition shows a kind of “peel off effect” after curing.

The dental cement composition also shows good sealing properties during the wearing time of a temporary restoration.

As the dental cement composition is based on (meth)acrylate components, the risk of incompatibilities to a later used permanent dental resin cement, which typically includes (meth)acrylate components as well, is reduced.

If a photo-initiator is present, an excess of the dental cement composition can also be removed more easily after use as the curing reaction of the dental cement composition can be triggered and further adjusted by the application of light.

Due to the presence of a high molecular polyol spacer with ether units between the (meth)acrylate moieties, the dental cement composition shows good flowing properties.

A good wetting behaviour may contribute to a better sealing of the tooth surface to which the dental cement composition has been applied.

Further, it was found that due to the use of a high molecular weight (meth)acrylate component a sufficient flexibility of the cured composition can already be achieved even if no or only minor amounts of softener are present.

The invention relates to a dental cement composition.

The dental cement composition is typically used for temporarily fixing a dental restoration to a dental situation in the mouth of a patient.

The dental cement composition comprises a resin matrix.

The dental cement composition comprises a (meth)acrylate component comprising a polyetherpolyol spacer group.

The (meth)acrylate component is typically present in the following amounts: Lower limit: at least 30 wt. % or at least 40 wt. % or at least 50 wt. %; Upper limit: utmost 80 wt. % or utmost 75 wt. % or utmost 70 wt. %; Range: 30 wt. % to 80 wt. % or 40 wt. % to 75 wt. % or 50 wt. % to 70 wt. %; wt. % with respect to the weight of the whole composition.

The (meth)acrylate component comprises at least two (meth)acrylate moieties.

According to one embodiment, the (meth)acrylate component may comprise not more than two (meth)acrylate moieties.

Using a (meth)acrylate with not more than two (meth)acrylate moieties may be advantageous as the crosslinking density of the network formed during the hardening reaction is lower compared to a situation where a (meth)acrylate component with three or more polymerizable moieties is used.

The (meth)acrylate component further comprises a polyol spacer group.

The polyol spacer group is preferably a polyether polyol and has a molecular weight Mw of at least 2,500 g/mol or at least 3,000 g/mol or at least 3,500 g/mol or at least 4,000 g/mol.

The molecular weight is typically in a range of 2,500 to 20,000 g/mol or 3,000 to 15,000 g/mol or 3,500 g/mol to 12,000 g/mol or 4,000 to 10,000 g/mol.

If desired, the molecular weight can be determined by titration of OH number of the starting OH-terminated polyether according to DIN EN ISO 4692-2. The molar mass can then be calculated by mathematically adding the molecular weight of the molecules used for chain extension and/or to introduce the (meth)acrylate functionality (e.g. isocyanato ethyl methacrylate).

Using a (meth)acrylate comprising preferably a polyether polyol spacer group having a molecular weight as described in the present text was found to be beneficial as it contributes to imparting elastomeric properties to the hardened dental cement composition.

The polyol spacer group may have the formula

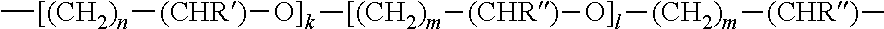

with
n=1 to 6, preferably 1 to 4, in particular 1, n being able to vary within the chain,
m=1 to 6, preferably 1 to 4, in particular 3, m being able to vary within the chain,
k, l=2 to 500, preferably 4 to 250, in particular 10 to 200,
R′, R″=H, methyl, ethyl, preferably R′=R″=H.

Preferred embodiments of the (meth)acrylate component include polyols with repeating units based on ethylene oxide, propylene oxide, tetramethylene oxide or mixtures therefrom.

The polyol spacer group may be produced by homo- or copolymerization of ethylene oxide, propylene oxide, tetrahydrofuran and/or ethylene oxide in the presence of strong acids, e.g. boron fluoride etherate.

Suitable polyol components are also commercially available, e.g. Acclaim™ Polyol 4200 (Covestro) or Acclaim™ Polyol 8200 (Covestro).

According to one embodiment, the (meth)acrylate component comprising a polyol spacer group may further include urethane groups and thus be regarded as a urethane (meth)acrylate component.

The urethane (meth)acrylate component may have a molecular weight (Mw) of at least 3,000 g/mol or at least 4,000 g/mol.

The molecular weight (Mw) is typically in a range of 3,000 to 22,0000 g/mol or 3,000 to 17,0000 g/mol or 4,000 to 15,0000 g/mol or 4,000 to 10,000 g/mol.

The urethane (meth)acrylate comprises at least two (meth)acrylate moieties.

The urethane (meth)acrylate comprises at least two urethane moieties.

According to one embodiment, the urethane (meth)acrylate does not comprise more than 6 or 4 or 2 urethane moieties.

It was found that with an increasing number of urethane moieties the hardened dental cement composition becomes more brittle and less elastic.

The (meth)acrylates according to the present invention may have the following structures:

MA-S2-L-S1-L-S2-MA (I)

MA-S2-L-(S1-U-S1)ₓ-L-S2-MA (II)

MA-S2-U-(S1-U-S1)ₓ-U-S2-MA (III)

with
MA being an acrylate or methacrylate moiety,
L being a urethane, an ester or an ether link,
U being a urethane moiety (O—CO—NH),
S1 being a polyetherpolyol having a molecular weight Mw of at least 2,500 g/mol,
S2 being a spacer group connecting a (meth)acrylate moiety and a urethane, an ester or ether moiety.
x being independently selected from 1 and 2, preferably 1.

The spacer groups S2 may comprise an C₂₋₆ alkyl chain. Particular preferred embodiments of the (meth)acrylate component are given in the example section.

The (meth)acrylate component can be produced by various reaction schemes.

According to one reaction scheme (structure I), the (meth)acrylate component is obtainable or is obtained by reacting preferably a polyetherpolyol component bearing two terminal OH-moieties as described in the present text with either a (meth)acrylate component having an isocyanate moiety, or a (meth)acrylic acid component, preferably an acid anhydride or acid chloride.

Another option (structure II) is by linking 2 polyetherpolyols bearing two terminal OH-moieties with a diisocyanate (molar ratio OH:NCO=2:1) and to react the remaining OH-groups with either a (meth)acrylate component having an isocyanate moiety, or a (meth)acrylic acid component, preferably an acid anhydride or acid chloride.

A further option (structure III) is by reacting a polyetherpolyol component bearing two terminal OH-moieties with an excess of a diisocanate (e.g. molar ratio OH:NCO=2:3) and to react the remaining NCO-groups with e.g. hydroxyethyl(meth)acrylate to generate a urethane(meth)acrylate. Examples are given in the experimental section.

The dental cement composition described in the present text comprises acidic polymerizable components. One or more polymerizable component(s) with acidic moiety(s) may be present, if desired.

The polymerizable components with an acid moiety can typically be represented by the following formula

AₙBCₘ
with A being an ethylenically unsaturated group, such as a (meth)acryl moiety,
B being a spacer group, such as (i) linear or branched C₁ to C₁₂ alkyl, optionally substituted with other functional groups (e.g. halogenides (including Cl, Br, I), OH or mixtures thereof) (ii) C₆ to C₁₂ aryl, optionally substituted with other functional groups (e.g. halogenides, OH or mixtures thereof), (iii) organic group having 4 to 20 carbon atoms bonded to one another by one or more ether, thioether, ester, thioester, thiocarbonyl, amide, urethane, carbonyl and/or sulfonyl linkages, and
C being an acidic group, or precursor of an acidic group such as acid anhydride,
m, n being independently selected from 1, 2, 3, 4, 5 or 6,
wherein the acidic group comprises one or more carboxylic acid residues, such as —COOH or —CO—O—CO—, phosphoric acid residues, such as —O—P(O)(OH)OH, phosphonic acid residues, such as C—P(O)(OH)(OH), sulfonic acid residues, such as —SO₃H or sulfinic acid residues such as —SO₂H.

Examples of polymerizable components with acid moiety include, but are not limited to glycerol phosphate mono(meth)acrylate, glycerol phosphate di(meth)acrylate, hydroxyethyl (meth)acrylate (e.g., HEMA) phosphate, bis((meth)acryloxyethyl) phosphate, (meth)acryloxypropyl phosphate, bis((meth)-acryloxypropyl) phosphate, bis((meth)acryloxy)propyloxy phosphate, (meth)acryloxyhexyl phosphate, bis((meth)acryloxyhexyl) phosphate, (meth)acryloxyoctyl phosphate, bis((meth)acryloxyoctyl) phosphate, (meth)acryloxydecyl phosphate, bis((meth)acryloxydecyl) phosphate, caprolactone methacrylate phosphate, citric acid di- or tri-methacrylate, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, poly(meth)acrylated polyboric acid, and the like. Derivatives of these hardenable components bearing an acid moiety that can readily react e.g. with water to form the specific examples mentioned above, like acid halides or anhydrides are also contemplated.

Also monomers, oligomers, and polymers of unsaturated carboxylic acids such as (meth)acrylic acids, aromatic (meth)acrylated acids (e.g., methacrylated trimellitic acids), and anhydrides thereof can be used.

Some of these compounds can be obtained, e.g., as reaction products between isocyanatoalkyl (meth)acrylates and carboxylic acids. Additional compounds of this type having both acid-functional and ethylenically unsaturated components are described in U.S. Pat. No. 4,872,936 (Engelbrecht) and U.S. Pat. No. 5,130,347 (Mitra). A wide variety of such compounds containing both the ethylenically unsaturated and acid moieties can be used. If desired, mixtures of such compounds can be used.

Using (meth)acrylate functionalized polyalkenoic acids is often preferred as those components were found to be useful to improve properties like adhesion to hard dental tissue, formation of a homogeneous layer, viscosity, or moisture tolerance.

According to one embodiment, the composition contains (meth)acrylate functionalized polyalkenoic acids, for example, AA:ITA:IEM (copolymer of acrylic acid:itaconic acid with pendent methacrylates).

These components can be made by reacting e.g. an AA:ITA copolymer with 2-isocyanatoethyl methacrylate to convert a portion of the acid groups of the copolymer to pendent methacrylate groups. Processes for the production of these components are described, e.g., in Example 11 of U.S. Pat. No. 5,130,347 (Mitra)); and those recited in U.S. Pat. No. 4,259,075 (Yamauchi et al.), U.S. Pat. No. 4,499,251 (Omura et al.), U.S. Pat. No. 4,537,940 (Omura et al.), U.S. Pat. No. 4,539,382 (Omura et al.), U.S. Pat. No. 5,530,038 (Yamamoto et al.), U.S. Pat. No. 6,458,868 (Okada et al.), and EP 0 712 622 A1 (Tokuyama Corp.) and EP 1 051 961 A1 (Kuraray Co., Ltd.).

If present, the polymerizable component(s) with acidic moiety(s) should be present in an amount so that the pH value of the composition is below 6, or below 4 or below 2, if brought in contact with water.

If present, the polymerizable component(s) with acidic moiety(s) is typically present in the following amounts: Lower limit: at least 3 wt. % or at least 5 wt. % or at least 8 wt. %; Upper limit: utmost 40 wt. % or utmost 30 wt. % or utmost 20 wt. %; Range: 3 wt. % to 40 wt. % or 5 wt. % to 30 wt. % or 8 wt. % to 20 wt. %; wt. % with respect to the weight of the whole composition.

The resin matrix further comprises a non-acidic polymerizable component with a molecular weight M_{w} below 1,000 g/mol.

The dental cement composition described in the present text further comprises a non-acidic polymerizable component, that is a polymerizable component without acidic moiety(s). One or more polymerizable component(s) without acidic moiety(s) may be present, if desired.

The non-acidic polymerizable component is typically a free-radically polymerizable material, including ethylenically unsaturated, monomers or oligomers or polymers.

Suitable polymerizable component(s) without acidic moiety(s) can be characterized by the following formula:

AₙBAₘ
with A being an ethylenically unsaturated group, such as a (meth)acryl moiety,
B being selected from (i) linear or branched C₁ to C₁₂ alkyl, optionally substituted with other functional groups (e.g. halogenides (including Cl, Br, I), OH or mixtures thereof) (ii) C₆ to C₁₂ aryl, optionally substituted with other functional groups (e.g. halogenides, OH or mixtures thereof), or (iii) organic group having 4 to 20 carbon atoms bonded to one another by one or more ether, thioether, ester, thioester, thiocarbonyl, amide, urethane, carbonyl and/or sulfonyl linkages,
m, n being independently selected from 0, 1, 2, 3, 4, 5 or 6 with the proviso that n+m is greater 0, that is that at least one A group is present.

Such polymerizable materials include mono-, di- or poly-acrylates and methacrylates such as methyl acrylate, methyl methacrylate, ethyl (meth)acrylate, isopropyl (meth)acrylate, n-hexyl (meth)acrylate, stearyl (meth)acrylate, allyl (meth)acrylate, glycerol di(meth)acrylate, the diurethane dimethacrylate called UDMA (mixture of isomers, e.g. Röhm Plex 6661-0) being the reaction product of 2-hydroxyethyl methacrylate (HEMA) and 2,2,4-trimethylhexamethylene diisocyanate (TMDI), glycerol tri(meth)acrylate, ethyleneglycol di(meth)acrylate, diethyleneglycol di(meth)acrylate, triethyleneglycol di(meth)acrylate, 1,3-propanediol diacrylate, 1,3-propanediol dimethacrylate, trimethylolpropane tri(meth)acrylate, 1,2,4-butanetrioltri(meth)acrylate, 1,4-cyclohexanediol di(meth)acrylate, pentaerythritol tri(meth)acrylate, pentaerythritol tetraacrylate, pentaerythritol tetramethacrylate, sorbitol hexa(meth)acrylate, bis[1-(2-(meth)acryloxy)]-p-ethoxyphenyldimethylmethane, bis[1-(3-methacryloxy-2-hydroxy)]-p-propoxyphenyldimethylmethane (BisGMA), bis[1-(3-acryloxy-2-hydroxy)]-p-propoxy-phenyldimethylmethane and trishydroxyethyl-isocyanurate trimethacrylate; the bis-acrylates and bis-methacrylates of polyethylene glycols of molecular weight 200-500, copolymerizable mixtures of acrylated monomers (see e.g. U.S. Pat. No. 4,652,274), and acrylated oligomers (see e.g. U.S. Pat. No. 4,642,126); and vinyl compounds such as styrene, diallyl phthalate, divinyl succinate, divinyl adipate and divinylphthalate; polyfunctional (meth)acrylates comprising urethane, urea or amide groups. Mixtures of two or more of these free radically polymerizable materials can be used if desired.

Further polymerizable components which may be present include di(meth)acrylates of ethoxylated bis-phenol A, for example 2,2′-bis(4-(meth)acryloxytetraethoxyphenyl)propanes. The monomers used can furthermore be esters of [alpha]-cyanoacrylic acid, crotonic acid, cinnamic acid and sorbic acid.

It is also possible to use the methacrylic esters mentioned in U.S. Pat. No. 4,795,823 (Gasser et al.), such as bis[3 [4]-methacryl-oxymethyl-8(9)-tricyclo[5.2.1.0^{2,6}]decylmethyl triglycolate. Suitable are also 2,2-bis-4(3-methacryloxy-2-hydroxypropoxy)phenylpropane (Bis-GMA).

These ethylenically unsaturated monomers can be employed in the dental composition(s) either alone or in combination with the other ethylenically unsaturated monomers.

Polymerizable monomers comprising a hydroxyl moiety and/or a 1,3-diketo moiety can also be added.

Suitable compounds include 2-hydroxyethyl (meth)acrylate (HEMA), 2- or 3-hydroxypropyl (meth)acrylate, 4-hydroxybutyl (meth)acrylate, 5-hydroxypentyl (meth)acrylate, 6-hydroxyhexyl (meth)acrylate, 10-hydroxydecyl (meth)acrylate, dialkylene glycol mono(meth)acrylate, for example, diethylene glycol mono(meth)acrylate, triethylene glycol mono(meth)acrylate, tetraethylene glycol mono(meth)acrylate, polyethylene glycol mono(meth)acrylate, dipropylene glycol mono(meth)acrylate, polypropylene glycol mono(meth)acrylate, and further 1,2- or 1,3- and 2,3-dihydroxypropyl (meth)acrylate, 2-hydroxypropyl-1,3-di(meth)acrylate, 3-hydroxypropyl-1,2-di(meth)acrylate, N-(meth)acryloyl-1,2-dihydroxypropylamine, N-(meth)acryloyl-1,3-dihydroxypropylamine, adducts of phenol and glycidyl (meth)acrylate, for example, 1-phenoxy-2-hydroxypropyl (meth)acrylate, 1-naphthoxy-2-hydroxypropyl (meth)acrylate, bisphenol A diglycidyl (meth)acrylate, 2-hydroxyethyl (meth)acrylate, 2-hydroxypropyl (meth)acrylate and 2,3-dihydroxypropyl (meth)acrylate are sometimes preferred.

An example of a polymerizable component with 1,3-diketo group is acetoacetoxy ethyl-methacrylate (AAEMA). If desired, mixtures of one or more of these components can be used.

Adding these components may be used to adjust the rheological properties or to influence mechanical properties.

The non-acidic polymerizable component is typically present in the following amounts: Lower limit: at least 2 wt. % or at least 5 wt. % or at least 8 wt. %; Upper limit: utmost 30 wt. % or utmost 25 wt. % or utmost 20 wt. %; Range: 2 wt. % to 30 wt. % or 5 wt. % to 24 wt. % or 8 wt. % to 20 wt. %; wt. % with respect to the weight of the whole composition.

The ratio of (meth)acrylate component to acidic polymerizable component is typically in a range of 2:1 to 20:1 with respect to wt. %. Using such a ratio can be advantageous as it allows a good balance between sufficient adhesion to the tooth structure and a high flexibility of the cement being the prerequisite to be removed in one piece.

The ratio of (meth)acrylate component to non-acidic polymerizable component is typically in a range of 1:1 to 20:1 with respect to wt. %. Using such a ratio can be advantageous as it may provide a good balance between the rheological and flow properties of the cement to ensure both wetting of the tooth and the restoration surface.

The ratio of acidic polymerizable component to non-acidic polymerizable component is typically in a range of 1:10 to 5:1 with respect to wt. %. Using such a ratio can be advantageous as it may enable a good balance between adhesion and mechanical properties.

The dental cement composition further comprises one or more fillers.

Adding a filler can be beneficial e.g. for adjusting the rheological properties like the viscosity. The content of the filler also typically influences the physical properties of the composition after hardening, like hardness or flexural strength.

The size of the filler particles should be such that a homogeneous mixture with the hardenable component forming the resin matrix can be obtained. The mean particle size of the filler may be in the range from 5 nm to 100 μm. If desired, the measurement of the particle size of the filler particles can be done as described in the example section.

The filler is typically present in the following amounts: Lower amount: at least 5 or at least 8 or at least 10 wt. %; Upper amount: utmost 55 or utmost 50 or utmost 45 wt. %; Range: 5 to 55 or 8 to 50 or 10 to 45 wt. %; wt. % with respect to the weight of the dental cement composition.

The filler(s) typically comprise non acid-reactive fillers. A non-acid reactive filler is a filler which does not undergo an acid/base reaction with an acid.

Useful non-acid reactive fillers include fumed silica, fillers based on non-acid reactive fluoroaluminosilicate glasses, quartz, ground glasses, non water-soluble fluorides such as CaF₂, silica gels such as silicic acid, in particular pyrogenic silicic acid and granulates thereof, cristobalite, calcium silicate, zirconium silicate, zeolites, including the molecular sieves.

Suitable fumed silicas include for example, products sold under the tradename Aerosil™ series OX-50, -130, -150, and -200, Aerosil™ R8200, R805 available from Evonik, CAB-O-SIL™ M5 available from Cabot Corp (Tuscola), and HDK types e.g. HDK™-H2000, HDK™ H15, HDK™ H18, HDK™ H20 and HDK™ H30 available from Wacker.

Filler(s) which can also be used and which provide radiopacity to the dental materials include heavy metal oxide(s) and fluoride(s). As used herein, “radiopacity” describes the ability of a hardened dental material to be distinguished from tooth structure using standard dental X-ray equipment in the conventional manner. Radiopacity in a dental material is advantageous in certain instances where X-rays are used to diagnose a dental condition. For example, a radiopaque material would allow the detection of secondary canes that may have formed in the tooth tissue surrounding a filling.

Oxides or fluorides of heavy metals having an atomic number greater than about 28 can be preferred. The heavy metal oxide or fluoride should be chosen such that undesirable colors or shading are not imparted to the hardened resin in which it is dispersed. For example, iron and cobalt would not be favoured, as they impart dark and contrasting colors to the neutral tooth color of the dental material. More preferably, the heavy metal oxide or fluoride is an oxide or fluoride of metals having an atomic number greater than 30. Suitable metal oxides are the oxides of yttrium, strontium, barium, zirconium, hafnium, niobium, tantalum, tungsten, bismuth, molybdenum, tin, zinc, lanthanide elements (i.e. elements having atomic numbers ranging from 57 to 71, inclusive), cerium and combinations thereof. Suitable metal fluorides are e.g. yttrium trifluoride and ytterbium trifluoride. Most preferably, the oxides and fluorides of heavy metals having an atomic number greater than 30, but less than 72 are optionally included in the materials of the invention. Particularly preferred radiopacifying metal oxides include lanthanum oxide, zirconium oxide, yttrium oxide, ytterbium oxide, barium oxide, strontium oxide, cerium oxide, and combinations thereof. The heavy metal oxide particles may be aggregated. If so, it is preferred that the aggregated particles are equal or less than 200 nm in average diameter.

Other suitable fillers to increase radiopacity are salts of barium and strontium especially strontium sulphate and barium sulphate.

Filler(s) which can also be used include nano-sized fillers such as nano-sized silica. Suitable nano-sized particles typically have a mean particle size in the range of 5 to 80 nm.

Preferred nano-sized silicas are commercially available from Nalco Chemical Co. (Naperville, Ill.) under the product designation NALCO™ COLLOIDAL SILICAS (for example, preferred silica particles can be obtained from using NALCO™ products 1040, 1042, 1050, 1060, 2327 and 2329), Nissan Chemical America Company, Houston, Texas (for example, SNOWTEX-ZL, -OL, -O, -N, -C, -20L, -40, and -50); Admatechs Co., Ltd., Japan (for example, SX009-MIE, SX009-MIF, SC1050-MJM, and SC1050-MLV); Grace GmbH & Co. KG, Worms, Germany (for example, those available under the product designation LUDOX™, e.g., P-W50, P-W30, P-X30, P-T40 and P-T40AS); Akzo Nobel Chemicals GmbH, Leverkusen, Germany (for example, those available under the product designation LEVASIL™, e.g., 50/50%, 100/45%, 200/30%, 200A/30%, 200/40%, 200A/40%, 300/30% and 500/15%), and Bayer MaterialScience AG, Leverkusen, Germany (for example, those available under the product designation DISPERCOLL™ S, e.g., 5005, 4510, 4020 and 3030).

Surface-treating the nano-sized silica particles before loading into the dental material can provide a more stable dispersion in the resin. Preferably, the surface-treatment stabilizes the nano-sized particles so that the particles will be well dispersed in the hardenable resin and results in a substantially homogeneous composition.

Thus, the silica particles as well as other suitable non acid-reactive fillers can be treated with a resin-compatibilizing surface treatment agent. The surface treatment or surface modifying agent include silane treatment agents. Especially preferred are silane treatment agents, which do not react with hardenable resins. Examples of silanes of this type include, for example, alkyl or aryl polyethers, alkyl, hydroxy alkyl, aryl, hydroxy aryl or amino functional silanes.

Non-acid reactive filler is typically present in the following amounts: Lower amount: at least 5 or at least 8 or at least 10 wt. %; Upper amount: utmost 50 or utmost 45 or utmost 40 wt. %; Range: 5 to 50 or 8 to 45 or 10 to 40 wt. %; wt. % with respect to the weight of the dental cement composition.

The dental cement composition may also contain an acid-reactive filler.

The presence of an acid-reactive filler may help to adjust the curing behaviour and adhesion of the dental cement composition by modulating the pH-value during the hardening process.

Acid-reactive fillers which can be used include metal oxides and hydroxides of e.g. calcium, magnesium or zinc, wherein the use of calcium hydroxide is sometimes preferred

The acid reactive filler is typically present in the following amounts: Lower amount: 0 or at least 0.1 or at least 0.2 wt. %; Upper amount: utmost 5 or utmost 4 or utmost 3 wt. %; Range: 0 to 5 or 0.2 to 4 or 0.3 to 3 wt. %; wt. % with respect to the weight of the dental cement composition.

The dental cement composition further comprises an initiator system. The initiator system is suitable for initiating a curing reaction of the polymerizable components of the dental cement composition.

The initiator system and its respective components are present in an amount sufficient to permit an adequate free-radical reaction rate. As the dental cement composition contains acidic components, the initiator system should be able to function in an acidic environment.

The initiator system may comprise a redox initiator system, a photo-initiator (system), or a combination of both.

According to one embodiment, the dental cement composition comprises a redox initiator system. Initiators, which rely upon a redox reaction, are often referred to as “auto-cure catalysts” or “dark cure catalysts”. To avoid a premature curing of the dental cement reaction, the two main components of this system (oxidizing and reducing agents) should be kept separate during storage of the dental cement composition.

As oxidizing components, typically peroxy components such as peroxides are used.

Organic peroxides which can be used include di-peroxides and hydroperoxides.

According to one embodiment, the organic peroxide is a di-peroxide, preferably a di-peroxide comprising the moiety R₁—O—O—R₂—O—O—R₃, with R₁ and R₃ being independently selected from H, alkyl (e.g. C₁ to C₆), branched alkyl (e.g. C₁ to C₆), cycloalkyl (e.g. C₅ to C₁₀), alkylaryl (e.g. C₇ to C₁₂) or aryl (e.g. C₆ to C₁₀) and R₂ being selected from alkyl (e.g. (C₁ to C₆) or branched alkyl (e.g. C₁ to C₆).

Examples of suitable organic di-peroxides include 2,2-Di-(tert.-butylperoxy)-butane and 2,5-Dimethyl-2,5-di-(tert-butylperoxy)-hexane and mixtures thereof.

According to another embodiment, the organic peroxide is a hydroperoxide, in particular a hydroperoxide comprising the structural moiety

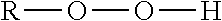

with R being (e.g. C₁ to C₂₀) alkyl, (e.g. C₃ to C₂₀) branched alkyl, (e.g. C₆ to C₁₂) cycloalkyl, (e.g. C₇ to C₂₀), alkylaryl (e.g. C₆ to C₁₂) or aryl (e.g. C₆ to C₁₂).

Examples of suitable organic hydroperoxides include t-butyl hydroperoxide, t-amyl hydroperoxide, p-diisopropylbenzene hydroperoxide, cumene hydroperoxide, pinane hydroperoxide, p-methane hydroperoxide and 1,1,3,3-tetramethylbutyl hydroperoxide and mixtures thereof.

Using hydroperoxides is sometimes preferred, in particular for formulating self-adhesive compositions.

Other peroxides which can be used are ketone peroxide(s), diacyl peroxide(s), dialkyl peroxide(s), peroxyketal(s), peroxyester(s) and peroxydicarbonate(s).

Examples of ketone peroxides include methyl ethyl ketone peroxide, methyl isobutyl ketone peroxide, methyl cyclohexanone peroxide, and cyclohexanone peroxide.

Examples of peroxyesters include alpha-cumylperoxyneodecanoate, t-butyl peroxypivarate, t-butyl peroxyneodecanoate, 2,2,4-trimethylpentylperoxy-2-ethyl hexanoate, t-amylperoxy-2-ethyl hexanoate, t-butylperoxy-2-ethyl hexanoate, di-t-butylperoxy isophthalate, di-t-butylperoxy hexahydroterephthalate, t-butylperoxy-3,3,5-trimethylhexanoate (TBPIN), t-butylperoxy acetate, t-butylperoxy benzoate and t-butylperoxymaleic acid.

Examples of peroxidicarbonates include di-3-methoxy peroxidicarbonate, di-2-ethylhexyl peroxy-dicarbonate, bis(4-t-butylcyclohexyl)peroxidicarbonate, diisopropyl-1-peroxydicarbonate, di-n-propyl peroxidicarbonate, di-2-ethoxyethyl-peroxidicarbonate, and diallyl peroxidicarbonate.

Examples of diacyl peroxides include acetyl peroxide, benzoyl peroxide, decanoyl peroxide, 3,3,5-trimethylhexanoyl peroxide, 2,4-dichlorobenzoyl peroxide and lauroylperoxide.

Examples of dialkyl peroxiodes include di-t-butyl peroxide, dicumylperoxide, t-butylcumyl peroxide, 2,5-dimethyl-2,5-di(t-butylperpoxy)hexane, 1,3-bis(t-butylperoxyisopropyl)benzene and 2,5-dimethyl-2,5-di(t-butylperoxy)-3-hexane.

Examples of peroxyketals include 1,1-bis(t-butylperoxy)-3,3,5-trimethylcyclohexane, 1,1-bis(t-butylperoxy)cyclohexane, 2,2-bis(t-butylperoxy)butane, 2,2-bis(t-butylperoxy)octane and 4,4-bis(t-butylperoxy)valeric acid-n-butylester.

If a peroxy component is present, it is typically present in an amount of 0.1 to 5 wt. % or 0.25 to 4 wt. % of the dental cement composition.

In addition to peroxide components, further oxidizing components can be present, such as persulfate components, in particular water-soluble persulfate components.

Persulfates which can be used can be characterized by the formula D₂S₂O₈ with D being selected from Li, Na, K, NH₄, NR₄, with R being selected from H and CH₃. Examples of persulfate(s) which can be used include, Na₂S₂O₈, K₂S₂O₈, (NH₄)₂S₂O₈ and mixtures thereof.

If a persulfate component is present, it is typically present in an amount of 0.1 to 5 wt. % or 0.25 to 4 wt. %.

As reducing agent barbituric acid or thiobarbituric acid components, in particular the respective salts thereof, can be used. Suitable barbituric acid components may be characterized by the following formula:

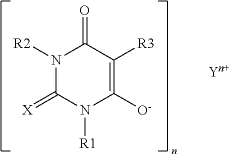

with R1, R2, and R3 being independently selected from hydrogen, alkyl, substituted alkyl, alkenyl, cycloalkyl, substituted cycloalkyl, arylalkyl, aryl or substituted aryl; X being oxygen or sulfur; Y being a metal cation or organic cation.

The salt may comprise a metal cation or inorganic cation. Suitable metal cations include any metals M that are able to provide stable cations M⁺, M²⁺, or M³⁺. Some possible inorganic cations include the cations of Li, Na, K, Mg, Ca, Sr, Ba, Al, Fe, Cu, Zn, or La.

Examples of suitable barbituric or thiobarbituric acid components include barbituric acid, thiobarbituric acid, 1,3,5-trimethylbarbituric acid, 1-phenyl-5-benzylbarbituric acid, 1-benzyl-5-phenyl-barbituric acid, 1,3-dimethylbarbituric acid, 1,3-dimethyl-5-phenylbarbituric acid, 1-cyclohexyl-5-ethylbarbituric acid, 5-laurylbarbituric acid, 5-butylbarbituric acid, 5-allylbarbituric acid, 5-phenylthiobarbituric acid, 1,3-dimethylthiobarbituric acid, trichlorobarbituric acid, 5-nitrobarbituric acid, 5-aminobarbituric acid, and 5-hydroxybarbituric acid.

An exemplary salt is the calcium salt of 1-benzyl-5-phenyl-barbituric acid. Another example of a suitable barbituric acid salt is the sodium salt of 1-benzyl-5-phenyl-barbituric acid. A possible salt is the calcium salt of 5-phenyl-thiobarbituric acid.

The salt may also comprise an organic cation. Suitable possible organic cations include the cations of amines, such as a cation of ammonium or a cation of alkylammonium. One example is the triethanolammonium salt of 1-benzyl-5-phenyl-barbituric acid.

If a barbituric acid or thiobarbituric acid component is present, it is typically present in an amount of 0.1 to 3 wt. % or 0.5 to 2 wt. % of the dental cement composition.

Other reducing agents which can be used include aromatic sulfinic acid salts or thiourea components. Suitable sulfinic acid components may have the formula

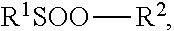

in which R¹ is an alkyl, substituted alkyl, alkenyl, cycloalkyl, substituted cycloalkyl, arylalkyl, aryl or substituted aryl radical and R²=H, metal such as lithium, sodium or potassium or is an alkyl, substituted alkyl, alkenyl, cycloalkyl, substituted cycloalkyl, arylalkyl, aryl or substituted aryl radical.

If one of the radicals R¹ or R² is unsubstituted alkyl then this radical can be straight-chain or branched and can contain, for example, from 1 to 18 carbon atoms, preferably from 1 to 10, and in particular from 1 to 6 carbon atoms. Examples of low-molecular alkyl radicals are methyl, ethyl, propyl, isopropyl, n-butyl, t-butyl, isobutyl, n-pentyl, and isoamyl.

If one of the radicals R¹ or R² is a substituted alkyl radical then the alkyl moiety of this radical typically has the number of carbon atoms indicated above for unsubstituted alkyl. If one of the radicals R¹ or R² is alkoxyalkyl or alkoxycarbonylalkyl then the alkoxy radical contains, for example, from 1 to 5 carbon atoms and is preferably methyl, ethyl, propyl, isopropyl, n-butyl, t-butyl, isobutyl, n-pentyl or isoamyl. If one of the radicals R¹ or R² is haloalkyl then the halo moiety is understood to be fluoro, chloro, bromo or iodo.

If one of the radicals R¹ or R² is alkenyl, then it is typically a C₃ to C₅ alkenyl radicals, especially allyl. If one of the radicals R¹ or R² is unsubstituted cycloalkyl, then it is typically C₄ to C₇ cycloalkyl radicals, such as cyclopentyl or cyclohexyl. If one of the radicals R¹ or R² is substituted cycloalkyl, then it is typically one of the above-indicated cycloalkyl radicals, with the substituent or substituents on the cycloalkyl radical possibly being, for example, C₁ to C₄ alkyl such as methyl, ethyl, propyl, n-butyl or isobutyl, fluoro, chloro, bromo, iodo or C₁ to C₄ alkoxy, especially methoxy. If one of the radicals R¹ or R² is aryl or aralkyl, then it is typically a phenyl or naphthyl as aryl. Preferred arylalkyl radicals include benzyl and phenylethyl.

R¹ or R² may also be substituted aryl radicals if desired. In this case phenyl and naphthyl are preferred and as ring substituents C₁ to C₄ alkyl, especially methyl, halogen or C₁ to C₄ alkoxy, especially methoxy.

In particular the following components were found to be useful: benzenesulfinic acid, sodium benzenesulfinate, sodium benzenesulfinate dihydrate, sodium toluenesulfinate, formamidinesulfinic acid, sodium salt of hydroxymethanesulfinic acid, sodium salt of 2,5-dichlorobenzenesulfinic acid, 3-acetamido-4-methoxybenzenesulfinic acid, wherein sodium toluenesulfinate or sodium benzenesulfinate and their hydrates are sometime preferred.

If a sulfinic acid component is present, it is typically present in an amount of 0.1 to 3 wt. % or 0.5 to 2 wt. % of the dental cement composition.

Suitable thiourea components include 1-ethyl-2-thiourea, tetraethyl thiourea, tetramethyl thiourea, 1,1-dibutyl thiourea, and 1,3-dibutyl thiourea and mixtures thereof.

If desired, the dental cement composition may contain a combination or mixture of different reducing agents, including a combination of barbituric acid components and sulfinic acid components.

Besides the above described components, the redox-initiator system may also comprise activators.

Suitable activators include, tertiary aromatic amines, such as the N,N-bis-(hydroxyalkyl)-3,5-xylidines (e.g. described in U.S. Pat. No. 3,541,068) as well as N,N-bis-(hydroxyalkyl)-3,5-di-t-butylanilines, in particular N,N-bis-([beta]-oxybutyl)-3,5-di-t-butylaniline as well as N,N-bis-(hydroxyalkyl)-3,4,5-trimethylaniline.

If desired and for acceleration, the polymerization can also be carried out in the presence of a transition metal component. Suitable transition metal component(s) include organic and/or inorganic salt(s) of vanadium, chromium, manganese, iron, cobalt, nickel, and/or copper, with copper, iron and vanadium being sometimes preferred.

According to one embodiment, the transition metal component is a copper containing component. The oxidation stage of copper in the copper containing component(s) is preferably +1 or +2.

Typical examples of copper component(s) which can be used include salts and complexes of copper including copper acetate, copper chloride, copper benzoate, copper acetylacetonate, copper naphthenate, copper carboxylates, copper bis(1-phenylpentan-1,3-dione) complex (copper procetonate), copper ethylhexanoate, copper salicylate, complexes of copper with thiourea, ethylenediaminetetraacetic acid and/or mixtures thereof. The copper compounds can be used in hydrated form or free of water.

Especially preferred are sometimes copper(II) acetate, copper bis(1-phenylpentan-1,3-dione) complex (copper procetonate), and copper ethylhexanoate.

According to one embodiment, the transition metal component is an iron containing component. The oxidation stage of iron in the iron containing component(s) is preferably +2 or +3.

Typical examples of iron containing component(s) which can be used include salts and complexes of iron including Fe(III) sulfate, Fe(III) chloride, iron carboxylates, iron naphthenate, Fe(III) acetylacetonate including the hydrates of these salts.

According to one embodiment, the transition metal component is a vanadium containing component. The oxidation stage of vanadium in the vanadium containing component(s) is preferably +4 or +5.

Typical examples of vanadium component(s) which can be used include salts and complexes of vanadium including vanadium acetylacetonate, vanadyl acetylacetonate, vanadyl stearate, vanadium naphthenate, vanadium benzoyl acetonate, vanadyl oxalate, bis(maltolato)oxovanadium (IV), oxobis(1-phenyl-1,3-butanedionate)vanadium (IV), vanadium (V) oxytriisopropoxide, ammon metavanadate (V), sodium metavanadate (V), vanadium pentoxide (V), divanadium tetraoxide (IV), and vanadyl sulfate (IV) and mixtures thereof, with vanadium acetylacetonate, vanadyl acetylacetonate, and bis(maltolato)oxovanadium (IV) being sometimes preferred.

Suitable redox initiator systems are also described in US 2003/008967 A1 (Hecht et al.), US 2004/097613 A1 (Hecht et al.), US 2019/000721 A1 (Ludsteck et al.). The content of these references is herewith incorporated by reference.

The dental cement composition may comprise a photo-initiator.

If the dental cement composition is provided as a kit of parts, the photo-initiator may be present in the base part or paste or the catalyst part or paste or in both parts or pastes. Typically, the photo-initiator is present in the catalyst part or paste.

The nature and structure of the photo-initiator is not particularly limited unless the intended purpose cannot be achieved. Suitable photo initiator(s) for free radical polymerization are generally known to the person skilled in the art dealing with dental materials.

As photo-initiator(s), those which can polymerize the polymerizable monomer(s) by the action of visible light having a wavelength of in the range of 350 nm to 500 nm are preferred.

Suitable photo-initiator(s) often contain an alpha di-keto moiety, an anthraquinone moiety, a thioxanthone moiety or benzoin moiety.

Examples of photo-initiator(s) include camphor quinone, 1-phenyl propane-1,2-dione, benzil, diacetyl, benzyl dimethyl ketal, benzyl diethyl ketal, benzyl di(2-methoxyethyl) ketal, 4,4,′-dimethylbenzyl dimethyl ketal, anthraquinone, 1-chloroanthraquinone, 2-chloroanthraquinone, 1,2-benzanthraquinone, 1-hydroxyanthraquinone, 1-methylanthraquinone, 2-ethylanthraquinone, 1-bromoanthraquinone, thio-xanthone, 2-isopropyl thioxanthone, 2-nitrothioxanthone, 2-methyl thioxanthone, 2,4-dimethyl thioxanthone, 2,4-diethyl thioxanthone, 2,4-diisopropyl thioxanthone, 2-chloro-7-trifluoromethyl thioxanthone, thioxanthone-10,10-dioxide, thioxanthone-10-oxide, benzoin methyl ether, benzoin ethyl ether, isopropyl ether, benzoin isobutyl ether, benzophenone, bis(4-dimethylaminophenyl)ketone, 4,4,′-bisdiethylaminobenzophenone.

Using acylphosphine oxides was found to be useful, as well.

Suitable acylphosphine oxides can be characterized by the following formula

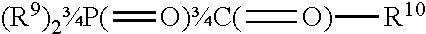

wherein each R⁹ individually can be a hydrocarbyl group such as alkyl, cycloalkyl, aryl, and aralkyl, any of which can be substituted with a halo-, alkyl- or alkoxy-group, or the two R⁹ groups can be joined to form a ring along with the phosphorous atom, and wherein R¹⁰ is a hydrocarbyl group, an S-, O-, or N-containing five- or six-membered heterocyclic group, or a —Z—C(═O)—P(═O)— (R⁹)₂ group, wherein Z represents a divalent hydrocarbyl group such as alkylene or phenylene having 2 to 6 carbon atoms.

Suitable systems are also described e.g. in U.S. Pat. No. 4,737,593 (Ellrich et al.), the content of which is herewith incorporated by reference.

Preferred acylphosphine oxides are those in which the R⁹ and R¹⁰ groups are phenyl or lower alkyl- or lower alkoxy-substituted phenyl. By “lower alkyl” and “lower alkoxy” is meant such groups having from 1 to 4 carbon atoms. In particular, 2,4,6-trimethylbenzoyl diphenyl phosphine oxide was found to be useful (Lucirin™ TPO, BASF).

More specific examples include: bis-(2,6-dichlorobenzoyl)phenylphosphine oxide, bis-(2,6-dichlorobenzoyl)-2,5-dimethylphenylphosphine oxide, bis-(2,6-dichlorobenzoyl)-4-ethoxyphenyl-phosphine oxide, bis-(2,6-dichlorobenzoyl)-4-biphenylylphosphine oxide, bis-(2,6-dichlorobenzoyl)-4-propylphenylphosphine oxide, bis-(2,6-dichlorobenzoyl)-2-naphthylphosphine oxide, bis-(2,6-dichlorobenzoyl)-1-napthylphosphine oxide, bis-(2,6-dichlorobenzoyl)-4-chlorophenylphosphine oxide, bis-(2,6-dichlorobenzoyl)-2,4-dimethoxyphenylphosphine oxide, bis-(2,6-dichlorobenzoyl)decylphosphine oxide, bis-(2,6-dichlorobenzoyl)-4-octylphenylphosphine oxide, bis-(2,6-dimethoxybenzoyl)-2,5-dimethylphenylphosphine oxide, bis-(2,6-dimethoxybenzoyl)phenylphosphine oxide, bis-(2,4,6-trimethylbenzoyl)-2,5-dimethylphenylphosphine oxide, bis-(2,6-dichloro-3,4,5-trimethoxybenzoyl)-2,5-dimethylphenylphosphine oxide, bis-(2,6-dichloro-3,4,5-trimethoxybenzoyl)-4-ethoxyphenylphosphine oxide, bis-(2-methyl-1-naphthoyl)-2,5-dimethylphenylphosphine oxide, bis-(2-methyl-1-naphthoyl)phenylphosphine oxide, bis-(2-methyl-1-naphthoyl)-4-biphenylylphosphine oxide, bis-(2-methyl-1-naphthoyl)-4-ethoxyphenylphosphine oxide, bis-(2-methyl-1-naphthoyl)-2-naphthyl-phosphine oxide, bis-(2-methyl-1-naphthoyl)-4-propylphenylphosphine oxide, bis-(2-methyl-1-naphthoyl)-2,5-dimethylphosphine oxide, bis-(2-methoxy-1-naphthoyl)-4-ethoxyphenylphosphine oxide, bis-(2-methoxy-1-naphthoyl)-4-biphenylylphosphine oxide, bis-(2-methoxy-1-naphthoyl)-2-naphthyl-phosphine oxide and bis-(2-chloro-1-naphthoyl)-2,5-dimethylphenylphosphine oxide.

The acylphosphine oxide bis(2,4,6-trimethylbenzoyl)phenyl phosphine oxide (previously known as IRGACURE™ 819 from Ciba Specialty Chemicals) is sometimes preferred.

Besides the photo-initiator, a reducing agent might be present. The combination of a photo-initiator and a reducing agent is often referred to as photo-initiator system.

As reducing agent or donor component, tertiary amines are generally used.

Suitable examples of the tertiary amines include N,N-dimethyl-p-toluidine, N,N-dimethyl-aminoethyl methacrylate, triethanolamine, methyl 4-dimethylaminobenzoate, ethyl 4-dimethyl-aminobenzoate, methyldiphenylamine and isoamyl 4-dimethylaminobenzoate.

If present, the photo-initiator is typically present in the following amounts: Lower amount: at least 0.01 or at least 0.02 or at least 0.03 wt. %; Upper amount: utmost 5 or utmost 4 or utmost 3 wt. %; Range: 0.01 to 5 or 0.02 to 4 or 0.03 to 3 wt. %; wt. % with respect to the weight of the dental cement composition.

The dental cement composition may further comprise one or more additives.

The composition described in the present text may also contain additives, such as dyes, pigments, photo-bleachable colorants, fluoride releasing agents, retarders, plasticisers solvents, etc.

Examples of dyes or pigments, which can be used include titanium dioxide or zinc sulphide (lithopones), red iron oxide 3395, Bayferrox™ 920 Z Yellow, Neazopon™ Blue 807 (copper phthalocyanine-based dye) or Helio™ Fast Yellow ER. These additives may be used for individual colouring of the dental compositions.

Examples of photo-bleachable colorants which can be present include Rose Bengal, Methylene Violet, Methylene Blue, Fluorescein, Eosin Yellow, Eosin Y, Ethyl Eosin, Eosin bluish, Eosin B, Erythrosin B, Erythrosin Yellowish Blend, Toluidine Blue, 4′,5′-Dibromofluorescein and blends thereof. Further examples of photobleachable colorants can be found in U.S. Pat. No. 6,444,725. The colour of the compositions of the invention may be additionally imparted by a sensitizing compound.

Examples of fluoride release agents which can be present include naturally occurring or synthetic fluoride minerals. These fluoride sources can optionally be treated with surface treatment agents.

Further additives, which can be added, include retarders, (such as 1,2-diphenylethylene), plasticizers (including polyethylene glycol derivatives, polypropylene glycols, low-molecular-weight polyesters, dibutyl, dioctyl, dinonyl and diphenyl phthalate, di(isononyl adipate), tricresyl phosphate, paraffin oils, glycerol triacetate, bisphenol A diacetate, ethoxylated bisphenol A diacetate, and silicone oils), flavorants, anti-microbials and/or fragrances.

Solvents, which can be present include linear, branched or cyclic, saturated or unsaturated alcohols, ketones, esters, ethers or mixtures of two or more of said type of solvents with 2 to 10 C atoms. Preferred alcoholic solvents include methanol, ethanol, iso-propanol and n-propanol. Other suitable organic solvents are THF, acetone, methyl ethyl ketone, cyclohexanol, toluene, alkanes and acetic acid alkyl esters, in particular acetic acid ethyl ester.

There is no need for these additive(s) to be present, so additive(s) might not be present at all. However, if present they are typically present in an amount which is not detrimental to the intended purpose.

The additive(s) is (are) typically present in the following amounts. Lower limit: 0 wt. % or at least 0.01 wt. % or at least 0.1 wt. %; Upper limit: utmost 20 wt. % or utmost 15 wt. % or utmost 10 wt. %; Range: 0 wt. % to 20 wt. % or 0.01 wt. % to 15 wt. % or 0.1 wt. % to 10 wt. %, wt. % with respect to the weight of the whole composition.

The dental cement composition may contain the respective components in the following amounts:

The dental cement composition may comprise, essentially consist of, or consist of
the (meth)acrylate component in an amount of 30 to 80 wt. %,
the acidic polymerizable component in an amount of 5 to 40 wt. %,
the non-acidic polymerizable component in an amount of 2 to 30 wt. %,
the filler in an amount of 5 to 55 wt. %,
the initiator system in an amount of 1 to 10 wt. %,
additives in an amount of 0 to 20 wt. %,
   wt. % with respect to the weight of the whole composition.

According to a further embodiment, the dental cement composition may comprise, essentially consist of, or consist of
the (meth)acrylate component in an amount of 40 to 70 wt. %,
the acidic polymerizable component in an amount of 10 to 40 wt. %,
the non-acidic polymerizable component in an amount of 5 to 30 wt. %,
the filler in an amount of 8 to 50 wt. %,
the initiator system in an amount of 1 to 10 wt. %,
additives in an amount of 0 to 20 wt. %,
   wt. % with respect to the weight of the whole composition.

The dental cement composition does typically not contain the following components alone or in combination: (meth)acrylate functionalized fillers in an amount of more than 5 wt. %; softener in an amount of more than 5 wt. %; polyhydric alcohol in an amount of more than 5 wt. %; eugenol in an amount of more than 0.1 wt. %; wt. % with respect to the dental cement composition.

Softener which are typically not present or are willfully added include phthalate, adipate, sebacate components and mixtures thereof.

Other softeners which are typically not present include gutta-percha, polyethylene, polypropylene, ethylene propylene copolymer, ethylene propylene terpolymer, silicone polymer, polyisoprene, ethylene vinyl acetate copolymer, and ethylene-(meth)acrylate copolymers such as ethylene-methyl (meth)acrylate copolymer, ethylene-ethyl (meth)acrylate copolymer and ethylene-butyl (meth)acrylate copolymer.

The dental cement composition may also be described with respect to its physical or mechanical properties.

The dental cement composition is acidic.

The dental cement composition may be characterized by the following properties before hardening: viscosity: 10 to 70 Pa*s at 28° C. and a shear rate of 10 s⁻¹ and pH value: 5 to 1.

For application, the dental cement composition is typically applied as a paste.

The paste has a viscosity which allows an easy application of the composition to a surface, e.g. by using an adequate mixing and delivering system. Suitable mixing and delivery systems are described below.

The dental cement composition may be characterized by the following properties alone or in combination after hardening: elongation at break: 20 to 50%; tensile strength: 4 to 10 MPa; shear bond strength (SBS) to polymerized (meth)acrylate material: 8 to 30 MPa; shear bond strength (SBS) to dentin: 0.2 to 3 MPa.

Tensile strength is a measurement of the force required to pull something to the point where it breaks. The hardened dental cement composition should have a sufficiently high tensile strength to ensure that the dental cement composition does not fracture once the dental restoration is removed from the dental situation in the mouth of a patient. A tensile strength in the mentioned range was found to be suitable.

An elongation at break in the mentioned range reflects the desired elastomeric properties of the dental cement composition. The composition should be sufficiently flexible and elastomeric.

The dental cement composition should also be sufficiently adhesive to a dental surface (e.g, surface of a prepared tooth), in particular to a dentine surface. A suitable method for determining the adhesion is the determination of the shear bond strength.

However, as the primary use of the dental cement composition is for temporarily fixing a dental restoration to a dental situation in the mouth of a patient, the adhesion should not be too strong. Otherwise, it might become difficult to remove a temporary dental restoration from the dental situation. A shear bond strength in the above range was considered sufficient and adequate.

For the intended use, the dental cement composition should also sufficiently adhere to the dental restoration to be fixed to the dental situation in the mouth of a patient.

As the adhesion of the dental cement to the dental restoration will depend on the material the dental restoration is made of (e.g., ceramic, metal, composite), the value of the adhesion will vary.

For achieving the desired result, the adhesion of the dental cement composition to the dental restoration should be higher than the adhesion to the dental tissue. This may help to ensure that most of the cement remains in the restoration once it is removed from the tooth surface, thus simplifying the process of cleaning the tooth surface prior to the permanent cementation of the final restoration.

Thus, the dental cement composition can also be characterized by the following equation:

Shear bond strength to restoration material>shear bond strength to dentin

The dental composition described in the present text is typically produced by combining or mixing the respective components, i.e. the polymerizable components of the resin matrix, the fillers and the initiator components together with other optional components, such as additives.

Mixing also includes kneading. If desired, a speed mixer can be used. Depending on the components to be mixed, the mixing is done under save light conditions.

The dental cement composition may be provided as a kit of part comprising a base part and a catalyst part.

The individual parts are typically kept separate during storage and are combined shortly before use.

The kit of parts may comprise a base part and a catalyst part,
the base part comprising,
   a portion of the (meth)acrylate component,
   the acidic polymerizable component,
   a portion of the non-acidic polymerizable component,
   a part of the initiator system, preferably an oxidizing component,
   a portion of the filler,
the catalyst part comprising,
   a portion of the (meth)acrylate component,
   a portion of the non-acidic polymerizable component,
   a part of the initiator system, preferably a reducing component,
   a portion of the filler,
      wherein the other components of the dental cement composition are present either in the base part or the catalyst part or the base part and the catalyst part and wherein the respective components are as described in the present text.

The viscosity of the base part and catalyst part are typically similar to facilitate the mixing process. The viscosity is typically in a range of 10 to 70 Pa*s at 28° C. measured at a shear rate of 10 s¹.

The dental cement composition is typically used in a process or for use in a process of temporarily restoring a dental situation in a mouth of a patient, the process comprising the steps of
providing a dental restoration, in particular a temporary dental restoration, and the dental cement composition described in the present text,
fixing the dental restoration with the aid of the dental cement composition to the dental situation to be restored,
removing the dental restoration from the dental situation.

By “temporarily restoring” is meant the restoration of a dental situation for a defined time period, which is typically the interim period until the permanent restoration is available (e.g. if made in a dental lab).

Such an interim period may range from a few days (e.g. 2 to 7 days) to a few weeks (e.g. 2 to 4 weeks) to a few months (e.g. 2 to 6 months).

The dental restoration may have different shapes, including the shape of a crown, bridge, inlay, onlay, veneer, or coping.

The dental restoration may be made of different materials, including composite, metal and ceramic or glass ceramic materials.

Typically, a temporary dental restoration is made of a composite material, in particular a bisacrylic composite or a PMMA material.

The invention is also directed to a kit of parts comprising the dental cement composition described in the present text and the following items alone or in combination: the dental cement composition described in the present text, a dental crown or a curable dental composition for producing a temporary dental restoration, the curable composition being different from the dental cement composition.

Suitable temporary dental restorative materials are described in US2011/053116A1 (Hecht et al.), U.S. Pat. No. 4,787,850 (Michl et al.), or US2006/229377A1 (Bublewitz et al.). The content of these references is herewith incorporated by reference.

Temporary dental restorative materials are also commercially available, e.g. Protemp™ 4 (3M Oral Care), Luxatemp® Automix Plus (DMG) or Integrity® (Dentsply).

The composition is typically stored in a packaging device before use.

Suitable packaging devices include cartridges, syringes and tubes. The volume of the packaging device used for storing is typically in the range of 0.1 to 100 ml or 0.5 to 50 ml or 1 to 30 ml.

A packaging device may also comprise two compartments, wherein each compartment is equipped with a nozzle for delivering the composition or parts stored therein. Once delivered in adequate portions, the parts can then be mixed by hand on a mixing plate.

The packaging device may have an interface for receiving a static mixing tip. The mixing tip is used for mixing the respective compositions.

The packaging device typically comprises two housings or compartments having a front end with a nozzle and a rear end and at least one piston movable in the housing or compartment.

Cartridges which can be used are also described e.g. in US2007/0090079A1 or U.S. Pat. No. 5,918,772, the disclosure of which is incorporated by reference. Some of the cartridges which can be used are commercially available e.g. from Sulzer Mixpac company (Switzerland).

Static mixing tips which can be used are described e.g. in US2006/0187752A1 or in U.S. Pat. No. 5,944,419, the disclosure of which is incorporated by reference. Mixing tips which can be used are commercially available from Sulzer Mixpac (Switzerland), as well.

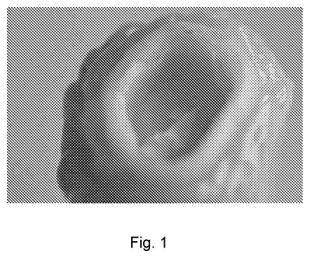

Other suitable storing devices are described e.g. in WO2010/123800 (3M), WO2005/016783 (3M), WO2007/104037 (3M), WO2009/061884 (3M), in particular the device shown in FIG. 14 of WO2009/061884 (3M) or WO2015/073246 (3M), in particular the device shown in FIG. 1 of WO2015/07346. Those storing devices have the shape of a syringe. The content of these references is herewith incorporated by reference, as well.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. The above specification, examples and data provide a description of the manufacture and use of the compositions and methods of the invention. The invention is not limited to the embodiments disclosed herein. One skilled in the art will appreciate that many alternative embodiments of the invention can be made without departing from the spirit and scope of thereof.

The following examples are given to illustrate the invention.

### EXAMPLES

Unless otherwise indicated, all parts and percentages are on a weight basis, all water is de-ionized water, and all molecular weights are weight average molecular weight. Moreover, unless otherwise indicated all experiments were conducted at ambient conditions (23° C.; 1013 mbar).

### Methods

### Viscosity

If desired, the viscosity of the mixed pastes can be determined by using a Physica MCR 301 Rheometer (Anton Paar, Graz, Austria) with a plate/plate geometry (PP08) at a constant shear rate of 10s⁻¹ in rotation at 28° C. The diameter of the plates is 8 mm and the gap between the plates is set to 0.75 mm. After mixing, about 200 mg of the mixture is placed on the cylindrical platform and the viscosity (in Pas) is determined. Each measurement should be performed twice.

### pH Value

If desired, the pH value of a composition can be determined as follows: A pH sensitive paper (Carl Roth™ company) is provided. A stripe of the pH sensitive paper is wetted. A small portion of the composition to be tested is placed on the wetted pH sensitive paper. After 5 s the colour change of the pH sensitive paper is determined.

### Particle Size Distribution

If desired, the particle size distribution can be determined by light-scattering, e.g. using the device Horiba (Horiba, JP).

The light scattering particle-sizer illuminates the sample with a laser and analyzes the intensity fluctuations of the light scattered from the particles at an angle of 173 degrees. The method of Photon Correlation Spectroscopy (PCS) can be used by the instrument to calculate the particle size. PCS uses the fluctuating light intensity to measure Brownian motion of the particles in the liquid. The particle size is then calculated to be the diameter of sphere that moves at the measured speed.

The intensity of the light scattered by the particle is proportional to the sixth power of the particle diameter. The Z-average size or cumulant mean is a mean calculated from the intensity distribution and the calculation is based on assumptions that the particles are mono-modal, mono-disperse, and spherical. Related functions calculated from the fluctuating light intensity are the Intensity Distribution and its mean. The mean of the Intensity Distribution is calculated based on the assumption that the particles are spherical. Both the Z-average size and the Intensity Distribution mean are more sensitive to larger particles than smaller ones.

The Volume Distribution gives the percentage of the total volume of particles corresponding to particles in a given size range. The volume-average size is the size of a particle that corresponds to the mean of the Volume Distribution. Since the volume of a particle is proportional to the third power of the diameter, this distribution is less sensitive to larger particles than the Z-average size. Thus, the volume-average will typically be a smaller value than the Z-average size. In the scope of this document the Z-average size is referred to as “mean particle size”.

### Shear Bond Strength (SBS-DE; SBS-MA)

The basis of the test method is a stainless steel rod with a defined surface area which is coated with the dental cement composition to be tested and then luted to the substrate (dentin or a methacrylate composite, e.g. Protemp 4™, 3M Oral Care) with standardized pressure. After curing the stainless steel rod will be sheared off in the shear test.

The details of the test method including the preparation of specimens, number of test specimens, curing (light-cure, dark-cure) and storing conditions are described in IADR/AADR Abstract ID #3318916.

All cements and were used according to manufacturers' instructions. Bovine teeth were ground flat to expose dentin or enamel, polished (grit 320 sandpaper), water-rinsed, and gently airdried.

Protemp 4™ test specimen (diameter 20.0 mm, height 3.5 mm) were hardened for 1 h at room temperature, then stored for 24 h±1 h at 36° C. in water, then ground flat (grit 320 sandpaper) and sandblasted (with alumina oxide particle size <50 μm).

Stainless steel rods (diameter=4 mm) were sandpapered, sandblasted, and silanized (ESPE™ Sil, 3M™ ESPE) and cemented (n=6) under standardized pressure (20 g/mm²). After excess removal, the cement was irradiated from 4 sides (10s each; Elipar™ S10, 3M™ ESPE). The specimens were stored for 10 min under pressure (36° C.) followed by additional 24h (36° C.; 100% relative humidity) without pressure.

If desired, the samples can be artificially aged (by thermocycling 5000 cycles 5° C.-55° C.) before shear bond strength testing (Zwick Z010; n=6; speed=0.75 mm/min).

The evaluation of average value and standard deviation was calculated by the computer of the Zwick machine. The type of failure is documented on the Zwick printout, as well as pretreatment, type of material, type of cylinder, batch number and test conditions.

### Tensile Strength (TS) and Elongation at Break (EB)

Tensile strength and elongation at break were determined essentially according to DIN ISO 527-1:2019 (Plastics) with the following modifications:

The test specimens were manufactured according to former DIN 53455 specimen type 4 with the following dimensions:

| Web: | Transition radii: | Total: |
|---|---|---|
| Height: 2.0 ± 0.1 mm | at the bridge: R = | Length: 25.0 ± 0.5 mm |
| | 14 ± 1 mm | |
| Width: 2.0 ± 0.1 mm | at the widening: R = | Broadening: |
| | 25 ± 2 mm | Width: 5.0 ± 0.1 mm |
| Length: 10.0 ± 0.1 mm | | |

The cured test specimens were stored at 100% humidity at 36° C. for 24 hours. The measurement was carried out on 6 test specimens, which were measured using a caliper before the test. The test specimens were pulled apart at a test speed of 2 mm/min until they break (using a Zwick test frame Z010). The software automatically determines tensile strength [MPa] and elongation at break [%].

### Crown Pull Off Test (CPOT)

Cleaned human teeth were coated at the root with 3M™ RelyX™ Unicem and then embedded in Versocit™ investment material up to approx. 2 mm below the enamel/dentine border. A tooth stump was prepared to the appropriate height and angle (6°) (4 mm). The shape of the tooth stump was digitally recorded using a dental scanner and read into a CAD/CAM software.

A composite crown was designed according to the scan data and manufactured by 3d-printed from an appropriate resin (e.g. the resin described in example 6 of WO 2018/231583 A1). The inner surface of the composite crown was pretreated by sandblasting with 50 μm alumina. The composite crown was cemented following a typical cementation protocol for temporary cements according to manufacturers' instructions. The restored tooth was stored for 24 hours at 36° C. and 100% humidity.

If desired, a thermocycling process can be conducted (wherein the temperature is switched between 5° C. and 55° C. for 5,000 cycles) followed by a chewing simulation (where a load of 50 N is applied 20,000 times) to simulate real time conditions in a patient's mouth.

Then the crown pull-off test was carried out with the aid of the Texture Analyzer TA HD Plus. where the composite crown was removed from the tooth stump. After removal the situation was visually inspected for failure mode and remaining cement residues.

### Evaluation:

++ essentially all (e.g. >80 wt. %) dental cement composition remained in composite crown;
+ >60% on composite crown
+/− 50% of dental cement remained in composite crown, 50% stays on tooth surface
− >60% on tooth surface
−− essentially all (e.g. >80 wt.) dental cement composition remained on tooth surface.

A picture of the inner surface of a composite crown which was temporarily cemented to a tooth surface with the dental cement composition of Example 1 and removed later is shown in FIG. 1.

After the pull-off test almost all of the dental cement composition (>90 wt. %) remained in the composite crown. Thus, the adhesion of the dental cement to the composite crown material was significantly higher than to the tooth structure.

### Materials

TABLE 1

| Compound | Abbreviation | Description |
|---|---|---|
| Glycerol dimethacrylate phosphate | GDMP | Adhesive monomer; Mw: 404 |
| | | g/mol |
| Triethylenglycole dimethacrylat | TEGDMA | Monomer; Mw: 286 g/mol |
| Diurethanedimethacrylate | Plex/UDMA | Monomer; Mw: 470 g/mol |
| Dodecanedioldimethylacrylate | DDDMA | Monomer Mw: 338.5 g/mol |
| Diol 6000 urethanedimethacrylate | DIOL-IEM | Monomer; Mw: about 6,300 |
| (Example 1) | | g/mol; Mw (polyol space): about |
| | | 6,000 g/mol |
| PPG-1000 extended with TMDI | PPG-1000- | Monomer; Mw: about 2,526 |
| (2 Mol:1 Mol) bis IEM-terminated | TMDI-MA | g/mol; Mw (polyol space): about |
| (Example 2) | | 1,000 g/mol |
| PPG-1000 bis IEM-terminated | PPG-1000- | Monomer; Mw: about 1,310 |
| (Example 3) | IEM-MA | g/mol; Mw (polyol space): about |
| | | 1,000 g/mol |
| PPG-2000 extended with TMDI | PPG-2000- | Monomer; Mw: about 4,710 |
| (2 Mol:1 Mol) bis IEM-terminated | TMDI-IEM- | g/mol; Mw (polyol space): about |
| (Example 4) | MA | 2,000 g/mol |
| Polypropylene-glycol-4200-bis-(2- | Acclaim-4200- | Monomer; Mw: about 4,510 |
| methacryloxyethylene)carbamate- | IEM | g/mol; Mw (polyol space): about |
| terminated | | 4,200 g/mol |
| (Example 5) | | |
| Diol 6000 dimethacrylate | DIOL-DMA | Monomer; Mw: about 6,130 |
| (Example 6) | | g/mol; Mw (polyol space): about |
| | | 6,000 g/mol |
| Aerosil ™ R 711 (surface treated fumed | Aerosil R 711 | Filler/rheological additive |
| silica) | | |
| HDK ™ H-2000 (surface treated fumed | HDK | Filler/rheological additive |
| silica) | | |
| Glass filler; silanated | GFS | Filler/Ion source |
| Calcium hydroxide | Ca(OH)₂ | Filler/Ion source |
| Tert. amylhydroperoxide | TAHP | Oxidizing agent |
| tert-Butylperoxy-3,5,5- | TBPIN | Oxidizing agent |
| trimethylhexanoat | | |
| Sodium persulphate | NaP | Oxidizing agent |
| 1-benzyl-5-phenyl barbituric acid (Ca- | Ca-BzPB | Reducing agent |
| salt) | | |
| Toluene sulfinic acid (Na-salt) | Na-TSS | Reducing agent |
| Copper acetate | CuAc | Accelerator |
| Copper procetonate | CuProc | Accelerator |
| Dibutyl-2-phenylethyl-ammonium | Chloride | Accelerator |
| chloride | | |
| Camphor quinone | CQ | Photoinitiator |
| Butylhydroxytoluene | Jonol | Stabilizer |

Preparation of Polyalkylene Oxide (cf. U.S. Pat. No. 6,677,393 B1)

The preparation of the polyalkylene oxide was done according to the preparation example in U.S. Pat. No. 6,677,393 B1 (columns 7/8).

### Formulas of Monomers

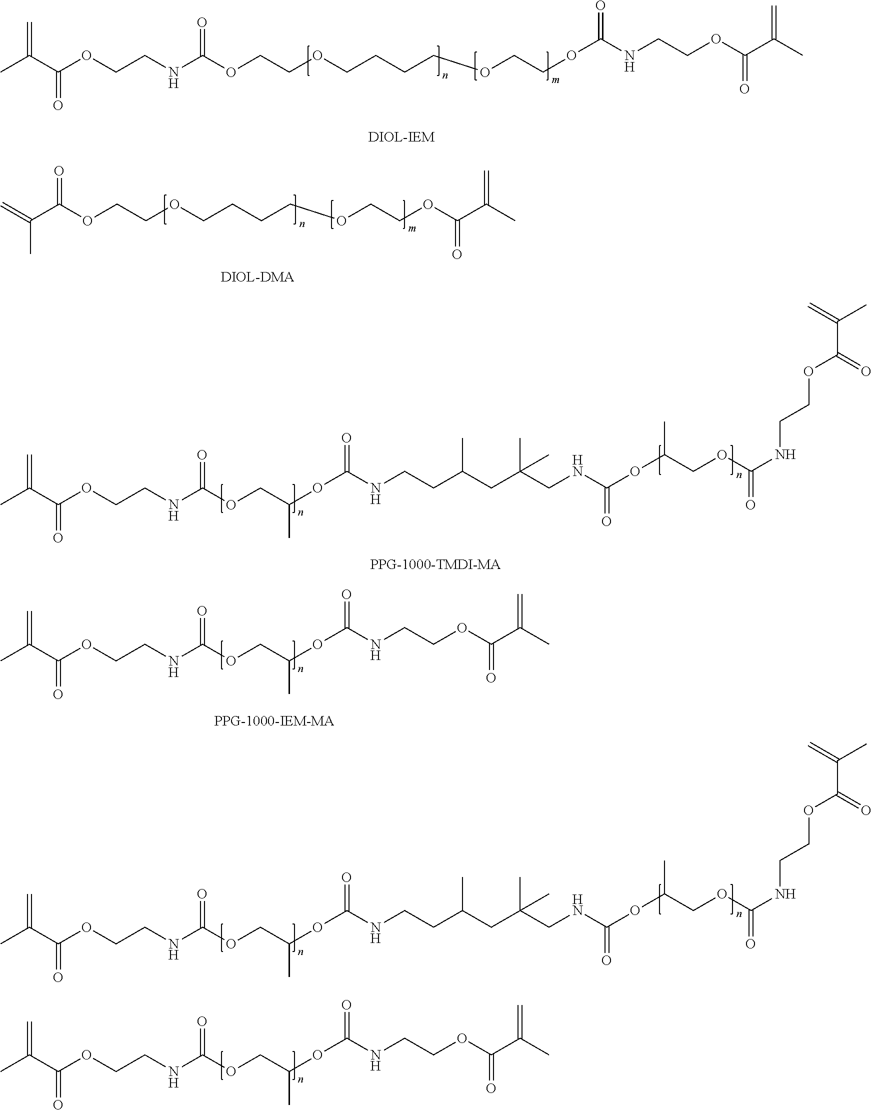

### PPG-2000-TMDI-IEM-MA

### Acclaim 4200-IEM

### General Procedure

The individual pastes were prepared by mixing the respective components under standardized conditions (room temperature, ambient pressure, 50% relative humidity) using a Hauschild Speedmixer (DAC 150 FVZ). The respective pastes were then mixed in a ratio of 1:1 by weight with a spatula on a mixing pad or with an automix system in a ratio of 1:1 by volume.

The curing was initiated either by mixing the pastes (auto cure) or by applying radiation (light cure). For light curing an Elipar™ S10 (3M Oral Care) device was used for 10 see each four sides. The obtained dental cement compositions were further analyzed with respect to their mechanical properties.

The following compositions were prepared:

### Example 1 (EX1; Inventive)

Catalyst Paste

| | wt. % |
|---|---|
| Ca(OH)₂ | 1.000 |
| Ca-BzPB | 4.000 |
| Na-TSS | 2.500 |
| Chloride | 0.382 |
| CQ | 0.150 |
| Jonol | 0.010 |
| Monomer-matrix | |
| DIOL-IEM | 86.458 |
| Filler | |
| R711 | 1.500 |
| HDK | 4.000 |

Base Paste

| | wt. % |
|---|---|
| TAHP | 1.136 |
| CuAC | 0.005 |
| Jonol | 0.010 |
| NaPS | 4.100 |
| Monomer-matrix | |
| GDMP | 30.290 |
| TEGDMA | 13.734 |
| DIOL-IEM | 36.994 |
| Filler | |
| R711 | 3.750 |
| HDK | 10.000 |

### Example 2 (EX2; Comparative)

Catalyst Paste

| | wt. % |
|---|---|
| Ca(OH)₂ | 1.000 |
| Ca-BzPB | 4.000 |
| Na-TSS | 2.500 |
| Chloride | 0.382 |
| CQ | 0.150 |
| Jonol | 0.010 |
| Monomer-matrix | |
| PPG-1000-TMDI-MA | 86.458 |
| Filler | |
| R711 | 1.500 |
| HDK | 4.000 |

Base Paste

| | wt. % |
|---|---|
| TAHP | 1.136 |
| CuAc | 0.005 |
| Jonol | 0.010 |
| NaPS | 4.100 |
| Monomer-matrix | |
| GDMP | 30.000 |
| PPG-1000-TMDI-MA | 50.728 |
| Filler | |
| R711 | 3.750 |
| HDK | 10.000 |

### Example 3 (EX3; Comparative)

Catalyst Paste

| | wt. % |
|---|---|
| Ca(OH)₂ | 1.000 |
| Ca-BzPB | 4.000 |
| Na-TSS | 2.500 |
| Chloride | 0.382 |
| CQ | 0.150 |
| Jonol | 0.010 |
| Monomer-matrix | |
| PPG-1000-IEM-MA | 86.458 |
| Filler | |
| R711 | 1.500 |
| HDK | 4.000 |

Base Paste

| | wt. % |
|---|---|
| TAHP | 1.136 |
| CuAc | 0.005 |
| Jonol | 0.010 |
| NaPS | 4.100 |
| Monomer-matrix | |
| GDMP | 30.000 |
| PPG-1000-IEM-MA | 50.728 |
| Filler | |
| R711 | 3.750 |
| HDK | 10.000 |

### Example 4 (EX4; Comparative)

Catalyst Paste

| | wt. % |
|---|---|
| Ca(OH)₂ | 1.000 |
| Ca-BzPB | 4.000 |
| Na-TSS | 2.500 |
| Chloride | 0.382 |
| CQ | 0.150 |
| Jonol | 0.010 |
| Monomer-matrix | |
| PPG-2000-TMDI-IEM-MA | 86.458 |
| Filler | |
| R711 | 1.500 |
| HDK | 4.000 |

Base Paste

| | wt. % |
|---|---|
| TAHP | 1.136 |
| CuAc | 0.005 |
| Jonol | 0.010 |
| NaPS | 4.100 |
| Monomer-matrix | |
| GDMP | 30.000 |
| PPG-2000-TMDI-IEM-MA | 50.728 |
| Filler | |
| R711 | 3.750 |
| HDK | 10.000 |

### Example 5 (EX5; Inventive)

Catalyst Paste

| | wt. % |
|---|---|
| Ca(OH)2 | 1.000 |
| Ca-BzPB | 4.000 |
| Chloride | 0.382 |
| Jonol | 0.010 |
| Monomer-matrix | |
| Acclaim-4200-IEM | 29.554 |
| Plex/UDMA | 17.732 |
| TEGDMA | 11.822 |
| Filler | |
| R711 | 1.500 |
| HDK | 4.000 |
| GFS | 30.000 |

Base Paste

| | wt. % |
|---|---|
| TAHP | 1.136 |
| CuAc | 0.005 |
| Jonol | 0.010 |
| Monomer-matrix | |
| GDMP | 10.272 |
| Acclaim-4200-IEM | 26.539 |
| Plex/UDMA | 15.923 |
| TEGDMA | 10.616 |
| Filler | |
| R711 | 4.000 |
| HDK | 1.500 |
| GFS | 30.000 |

### Example 6 (Inventive)

Catalyst Paste

| | wt. % |
|---|---|
| Ca(OH)2 | 1.000 |
| Ca-BzPB | 4.000 |
| Chloride | 0.382 |
| Jonol | 0.010 |
| Monomer-matrix | |
| Diol-DMA | 47.286 |
| DDDMA | 11.822 |
| Filler | |
| R711 | 1.500 |
| HDK | 4.000 |
| GFS | 30.000 |

Base Paste

| | wt. % |
|---|---|
| TBPIN | 0.600 |
| CuProc | 0.019 |
| Jonol | 0.010 |
| D-Zethacrylat | 2.981 |
| Monomer-matrix | |
| GDMP | 5.000 |
| DDDMA | 11.178 |
| Diol-DMA | 44.712 |
| Filler | |
| R711 | 1.500 |
| HDK | 4.000 |
| GFS | 30.000 |

### Results

TABLE 2

| | SBS-DE | SBS-DE | SBS-MA | SBS-MA | | | |
|---|---|---|---|---|---|---|---|
| | (MPa) | (MPa) | (MPa) | (MPa) | TS | EB | |
| | Light cure | Auto cure | Light cure | Auto cure | (MPa) | (%) | CPOT |
| EX1 | 2.58 | n.m. | 13.43 | 16.72 | 4.67 | 38.2 | ++ |
| | (0.73) | | (2.18) | (3.26) | (0.88) | (9.7) | |
| EX2 | n.m. | n.m. | n.m. | n.m. | 3.74 | 18.8 | n.m. |
| | | | | | (0.56) | (4.3) | |
| EX3 | n.m. | n.m. | n.m. | n.m. | 3.80 | 11.20 | n.m. |
| | | | | | (0.55) | (1.8) | |
| EX4 | n.m. | n.m. | n.m. | n.m. | 3.25 | 28.0 | n.m. |
| | | | | | (0.42) | (5.5) | |
| EX5 | n.m. | 0.18 | n.m. | 22.69 | n.m. | n.m. | n.m. |
| | | (0.28) | | (2.78) | | | |
| EX6 | n.m. | n.m. | n.m. | n.m. | 7.50 | 33.6 | n.m. |
| | | | | | (1.24) | (5.6) | |

standard deviation is given in brackets;
n.m. = not measured

As can be taken from the measured data, the inventive dental cement compositions show a higher flexibility compared to the comparative examples (Examples 2-4).

1. A dental cement composition comprising
a resin matrix comprising
a (meth)acrylate component comprising
at least two (meth)acrylate moieties,
a polyol spacer group between the at least two (meth)acrylate moieties, the polyols spacer group having a molecular weight Mw of at least 3,000 g/mol,
an acidic polymerizable component, preferably an acidic (meth)acrylate component,
a non-acidic polymerizable component having a molecular weight Mw below 1,000 g/mol, preferably a non-acidic (meth)acrylate component,
filler,
an initiator system,
optionally additives.

2. The dental cement composition according to claim 1, the (meth)acrylate component having either of the following structures:
MA-S2-L-S1-L-S2-MA (I)
MA-S2-L-(S1-U-S1)ₓ-L-S2-MA (II)
MA-S2-U-(S1-U-S1)ₓ-U-S2-MA (III)
with
MA being an acrylate or methacrylate moiety,
L being a urethane, an ester or an ether link,
U being a urethane moiety,
S1 being a polyol moiety, preferably a polyether polyol, having a molecular weight Mw of at least 3,000 g/mol,
S2 being a spacer group connecting a (meth)acrylate moiety and a urethane, an ester or ether moiety,
x being 1 to 2.

3. The dental cement composition according to claim 2,
the spacer groups S2 comprising an C₂₋₆ alkyl chain;
the spacer group S1 comprising a polyol chain having the formula
—[(CH₂)ₙ—(CHR′)—O]ₖ—[(CH₂)ₘ—(CHR″)—O]ₗ—(CH₂)ₘ—(CHR″)—
with
n=1 to 6, preferably 1 to 4, in particular 1, n being able to vary within the chain,
m=1 to 6, preferably 1 to 4, in particular 3, m being able to vary within the chain,
k, l=2 to 500, preferably 4 to 250, in particular 10 to 200,
R′, R″=H, methyl, ethyl, preferably R′=R″=H.

4. The dental cement composition according to claim 1, the (meth)acrylate component being a urethane (meth)acrylate component comprising at least two (meth)acrylate moieties, and not more than 4 or not more than 2 urethane moieties.

5. The dental cement composition according to claim 1, characterized by the following features alone or in combination:
the ratio of (meth)acrylate component to acidic polymerizable component being in a range of 2:1 to 20:1 with respect to wt. %;
the ratio of (meth)acrylate component to non-acidic polymerizable component being in a range of 1:1 to 20:1 with respect to wt. %;
the ratio of acidic polymerizable component to non-acidic polymerizable component being in a range of 1:10 to 5:1 with respect to wt. %.

6. The dental cement composition according to claim 1, the initiator system comprising a redox initiator system, a photo-initiator, or a combination of both.

7. The dental cement composition according to claim 1, the filler comprising non-acid reactive fillers.

8. The dental cement composition according to claim 1 comprising
the (meth)acrylate component in an amount of 30 to 80 wt. %,
the acidic polymerizable component in an amount of 5 to 40 wt. %,
the non-acidic polymerizable component in an amount of 2 to 30 wt. %,
the filler in an amount of 5 to 55 wt. %,
the initiator system in an amount of 1 to 10 wt. %,
additives in an amount of 0 to 20 wt. %,
wt. % with respect to the weight of the whole composition.

9. The dental cement composition according to claim 1 not comprising the following components alone or in combination:
(meth)acrylate functionalized fillers in an amount of more than 5 wt. %;
softener in an amount of more than 5 wt. %;
polyhydric alcohol in an amount of more than 5 wt. %,
eugenol in an amount of more than 0.1 wt. %;
wt. % with respect to the dental cement composition.

10. The dental cement composition according to claim 1, the (meth)acrylate component being obtainable by reacting a polyol component having a molecular weight Mw of at least 3,000 g/mol with either a (meth)acrylate component having an isocyanate moiety, or a (meth)acrylic acid component, preferably an acid anhydride or acid chloride.

11. The dental cement composition according to claim 1 being characterized by the following features alone or in combination before hardening:
viscosity: 10 to 70 Pa*s at 28° C. and a shear rate of 10 s⁻¹;
pH value: 5 to 1.

12. The dental cement composition according to claim 1 being characterized by the following features alone or in combination after hardening:
elongation at break: 20 to 50%;
tensile strength: 4 to 10 MPa;
shear bond strength to polymerized (meth)acrylate material: 8 to 30 MPa;
shear bond strength to dentin: 0.1 to 3 MPa.

13. The dental cement composition according to claim 1 being provided as a kit of parts comprising a base part and a catalyst part,
the base part comprising,
a portion of the (meth)acrylate component,
the acidic polymerizable component,
a portion of the non-acidic polymerizable component,
a part of the initiator system, preferably an oxidizing component,
a portion or part of the filler
the catalyst part comprising,
a portion of the (meth)acrylate component,
a portion of the non-acidic polymerizable component,
a part of the initiator system, preferably a reducing component,
a portion or part of the filler
wherein the other components of the dental cement composition are present either in the base part or the catalyst part or the base part and the catalyst part.

14. A dental cement composition for use in a process of temporarily restoring a dental situation in a mouth of a patient, the process comprising the steps of
providing a dental restoration and the dental cement composition as described in claim 1
fixing the dental restoration with the aid of the dental cement composition to the dental situation to be restored,
removing the dental restoration from the dental situation.

15. A kit of parts comprising the dental cement composition as described in claim 1 and a dental crown or a curable dental composition for producing a temporary dental restoration being different from the dental cement composition.
